(19) United States
(12) Patent Application Publication
(10) Pub. No.: US 2004/0073256 A1
(43) Pub. Date: Apr. 15, 2004

(54) Activated surgical fasteners, devices therefor and uses thereof

(76) Inventors: Kevin Marchitto, Golden, CO (US); Stephen T. Flock, Arvada, CO (US)

Correspondence Address: Benjamin Aaron Adler, ADLER & ASSOCIATES, 8011 Candle Lane, Houston, TX, 77071, US

(21) Appl. No.: 10/637,383

(22) Filed: Aug. 8, 2003

Related U.S. Application Data
(60) Provisional application No. 60/438,752, filed on Jan. 8, 2003.
(60) Provisional application No. 60/402,524, filed on Aug. 9, 2002.

(51) Int. Cl. A61B 17/08

(52) U.S. Cl. 606/219

(57) ABSTRACT

Provided herein is an activated surgical fastener to effect a weld between the substrates in an individual. The surgical fastener comprises a means for attaching the substrates and a fusion composition which welds the substrates upon the application of energy thereto. Also provided is a surgical device comprising the surgical fastener, an applicator, a means of delivering energy to the fastener and a means to control the welding process. Additionally, methods of surgically fastening at least two substrates are provided.

### CROSS-REFERENCE TO RELATED APPLICATIONS

This non-provisional application claims benefit of U.S. provisional 60/438,752, filed Jan. 8, 2003, now abandoned and U.S. provisional 60/402,524, filed Aug. 9, 2002, now abandoned.

### BACKGROUND OF THE INVENTION

1. Field of the Invention

The present invention relates generally to the fields of biomedical physics and wound closure. More specifically, the present invention provides devices and methods for closing a wound following a surgical procedure.

2. Description of the Related Art

Conventional methods of wound closure following surgery consist of applying sutures or staples to join two or more tissues that have been dissected. While these methods are generally successful, at times complications arise due to inadequate closure of the wound that could result in the tissues separating or in “leakiness.” In particular, the quality of suturing depends on manual dexterity of the surgeon and adequate access to the wound. Current designs of surgical clips can slip if applied incorrectly or accidentally disturbed. Surgical clips can also cause damage to the vessels or structures to which they are applied if the surgeon applies excessive compression force. With the increasing use of minimally invasive surgical methods, such as endoscopy, wound access and the efficient closure of wounds has become a significant issue in medicine.

A surgeon's skill is less of a factor where surgical staples are employed and, as a result, less invasive devices have been developed for effective delivery of staples through endoscopic trocars. This has led to greater acceptance for stapling devices over suturing during less invasive surgical procedures. Nonetheless, conventional stapling is limited in that it usually requires an anvil be placed behind the tissues to be joined, and that enough space is available to produce the necessary force to form the staple against the anvil.

Various methods have been employed to fasten tissues together without the use of a conventional staple or suture. These devices often employ springs or another compression mechanism to pull the tissues together. Shape memory alloys have been employed in U.S. Pat. No. 4,485,816, U.S. Pat. Nos. 5,002,562 and 6,113,611 and, in at least one case, using electronic heating of the fastener to make it close. U.S. Pat. No. 5,725,522 discloses the employment of lasers to effect suture “fusion” whereby two ends of the suture are fused together in place of the traditional knot.

More recently, wound sealing approaches, which employ methods of directing energy to the tissue which as a consequence adheres to proximal tissue, have been tested and used clinically. Commercial electrosurgery and electrocautery devices are commonly used for sealing internal wounds. Inventions for sealing vessels using other forms of electromagnetic energy have been published. For example U.S. Pat. No. 6,033,401 describes a device to deliver adhesive and apply microwave energy to effect sealing of a vessel. In U.S. Pat. No. 6,179,834 a vascular sealing device to provide a clamping force during which radiofrequency energy is applied until a particular temperature or electrical impedance is reached is described. Further, U.S. Pat. No. 6,132,429 describes using a radiofrequency device to weld blood vessels closed and monitoring the process by changes in tissue temperature or electrical impedance.

A trend toward the use of minimally invasive surgical techniques has created a demand for wound closure methodology that can be used through a small incision in the patient. Sutures cannot easily be secured by traditional methods through an endoscope and current stapling methods generally require an anvil be placed behind the tissue thereby limiting their use. U.S. Pat. No. 6,358,271 describes the use of sutures composed of a fused loop of filamentous material which is ultrasonically welded. This application has the advantage of a low profile of suture closure as compared to the traditional knot and may ultimately be applied endoscopically, however the technology still requires the use of a fairly large securing device including an anvil. U.S. Pat. Nos. 6,409,743 and 6,423,088 discuss c-shaped collars that are made out of a material that fuses to itself upon the application of enegy in the form of heat, light, radiofrequency waves, electricity or ultrasound.

There has been an effort recently to identify biocompatible molecules which can be used as a “tissue solder”. Biomolecules such as fibrin, elastin or albumin have been, or are being used to “glue” tissue-to-tissue. A number of patents describe the activation of these biomolecules with radiant energy to form tissue welds with the energy often being in the form of laser radiation, or, sometimes, in the form of ultrasound or radiofrequency waves. The applied energy is believed to denature the molecules which then adhere to one-another or cross-link upon renaturation to effect a bond.

U.S. Pat. No. 5,669,934 discloses a method for joining or restructuring tissue consisting of providing a preformed film or sheet of a collagen and/or gelatin material that fuses to tissue upon the application of continuous inert gas beam radiofrequency energy. Similarly, U.S. Pat. No. 5,569,239 describes laying down a layer of energy reactive adhesive material along the incision and closing the incision by applying either optical energy or radiofrequency energy to the adhesive and surrounding tissue. Furthermore, U.S. Pat. Nos. 5,209,776 and 5,292,362 describe a tissue adhesive that is principally intended to be used in conjunction with laser radiant energy to weld severed tissues and/or prosthetic material together.

U.S. Pat. No. 6,110,212 describes the use of elastin and elastin-based materials which are biocompatible and can be used to effect anastomoses and tissue structure sealing upon the application of laser radiant energy. The stated benefits, inter alia, are the biocompatible and ubiquitous nature of elastin. U.S. patent application No. 20020198517 discloses the use of laser tissue-welding employing an adhesive consisting mostly of gelatin which effects tissue attachment.

Further, U.S. Pat. No. 6,302,898 describes a device to deliver a sealant and energy to effect tissue closure. The tissue is pre-treated with energy in order to make the subsequently applied sealant adhere better. In PCT Appplication No. WO 99/65536 tissue repair by pre-treating the substantially solid biomolecular solder prior to use is taught.

U.S. Pat. No. 5,713,891 describes the addition of bioactive compounds to the tissue solder in order to enhance the weld strength or to reduce post-procedure hemorrhage. U.S. Pat. No. 6,221,068 discloses the importance of minimizing thermal damage to the tissue to be welded. By using pulsed laser radiation and allowing the tissue to cool to nearly the initial temperature between each heating cycle, the damage is minimized.

U.S. Pat. No. 6,323,037 describes the addition of an “energy converter” to the solder mixture such that incident optical energy will be efficiently and preferentially absorbed by the solder which subsequently effects a tissue weld. Similarly U.S. Pat. No. 6,348,679 describes using a radiofrequency “susceptor”, i.e., a compound that absorbs RF energy and converts it to heat.

Common problems exist throughout the prior art. These include tissue damage due to uneven heating, unknown and/or uncontrollable thermal history, i.e., temperature as a function of time, and relatively high cost. It is notable that a consistent means of treatment and control is recognized to be desirable. The Code of Federal Regulations at 21 CFR 860.7(e)(1) establishes that there is “reasonable assurance that a device is effective when it can be determined, based upon valid scientific evidence, that in a significant portion of the target population, the use of the device will provide clinically significant results.” Devices that cannot be shown to provide consistent results between patients or even within a patient upon multiple use will have minimal utility and may not be approvable for broad use. Beyond devices it is generally desirable to develop medical products with critical controls that can deliver a precise result.

The inventors have recognized a need in the art for a precision device and improved methods of joining tissues which have been separated through surgery or through trauma, particularly during minimally invasive procedures. The prior art is particularly deficient in devices and minimally-invasive methods that use electromagnetic energy to controllably alter a biocompatible structure thereby making it adhere to tissue through molecular alterations and/or mechanical shrinkage. The present invention fulfills this longstanding need and desire in the art.

### SUMMARY OF THE INVENTION

The present invention is directed to an activated surgical fastener to effect a weld between at least two substrates in an individual comprising a means for attaching the substrates and a fusion composition to weld the attached substrates upon the application of energy thereto. The activated surgical fastener may comprise a surgical suture or a staple, pin or clip.

The present invention also is directed to a surgical device comprising the activated surgical fastener described herein, an applicator to position at least one of the fastener in relation to at least two substrates, a means of delivering energy to the fastener to effect a weld between the substrates, and a means to control the welding process.

The present invention is directed further to a method of surgically fastening at least two substrates. The activated surgical fastener described herein is positioned in relation to the substrates with the surgical device, also described herein, to attach the substrates. A weld is formed between the activated surgical fastener and the substrates with the surgical device thereby surgically fastening the substrates.

Other and further aspects, features, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others that will become clear, are attained and can be understood in detail, more particular descriptions of the invention briefly summarized above may be had by reference to certain embodiments thereof that are illustrated in the appended drawings. These drawings form a part of the specification. It is to be noted, however, that the appended drawings illustrate preferred embodiments of the invention and therefore are not to be considered limiting in their scope.

FIG. 1 depicts a closed loop surgical suture composed of filamentous material and a metal.

FIG. 2 depicts surgical suture composed of filamentous material and handheld applicator to effect a weld in the suture along the lengths of suture in contact with one another.

FIG. 3 depicts a cross-sectional view of an applicator that may be used to hold the ends of a single suture, or two connecting suture ends, in proximity to one another while inductively heating the material of the suture or applicator.

FIG. 4 depicts a cross-sectional view of the applicator of FIG. 3 to hold a ridged suture material inserted into the element in place. A second ridged suture, or the end of the same suture is placed in the other end.

FIG. 5 depicts an applicator that may be used to inductively heat the suture.

FIG. 6 depicts a surgical pin for placement between two sections of overlapping tissue.

FIG. 7 depicts a surgical staple to fasten tissue that has been separated.

FIG. 8A depicts a surgical compression staple.

FIG. 8B depicts a multi-piece surgical compression staple.

FIG. 9 depicts a tissue-fastening device with a single layer of material having angular spines on one surface only.

FIG. 10 depicts a compression style tissue-fastening device, with multiple layers of material with different shape conforming qualities having angular spines along an inner surface.

FIG. 11 depicts a two-sided compression style tissue-fastening device composed partly of a conductive, shrinkable material and having angular spines along an inner surface.

FIG. 12 depicts an applicator for delivering tissue-fastening devices.

### DETAILED DESCRIPTION OF THE INVENTION

In one embodiment of the present invention there is provided an activated surgical fastener to effect a weld between at least two substrates in an individual comprising a means for attaching the substrates and a fusion composition to weld the attached substrates upon the application of energy thereto.

In all aspects of this embodiment the energy may be applied conductively or inductively to the fusion composition. The energy applied to the fusion composition may be radiofrequency energy, radiant energy or microwave energy. A preferable form of energy is radiofrequency energy when induction is used.

Again in all aspects, the fusion composition may be a biocompatible material. The fusion composition may be a protein, a metal, a ferromagnetic material, a conducting or non-conducting polymer, a pharmaceutical, an ionic mixture or a combination thereof. At least one of the to be fastened may be a biological tissue. The tissue may have been surgically separated or may be surgically grafted.

Still in all aspects of this embodiment the activated surgical fastener is positioned in relation to the substrates by an applicator. An example of an applicator utilizes pressure to position the surgical fastener. In such an applicator, the pressure may be created with a spring mechanism or with a gas. Additionally, the applicator may be used endoscopically.

Further to all aspects of this embodiment, the activated surgical fastener may comprise at least one conductive element. The conductive element may be a conductive electrode or an electrode array. The electrode array has a plurality of isolated electrode terminals. The conductive element also may be heated inductively.

Additionally, the activated surgical fastener may comprise at least one material that changes configuration upon the application of energy thereto. Examples of the material are a shape-memory polymer or a shape-memory metal. The material may shrink upon heating.

In a specific aspect of this embodiment the attaching means is a surgical suture comprising a filamentous material having a first end and a second end such that the first and second ends are juxtaposed to form a closed loop for attaching the substrates. The ends are welded upon application of energy to the fusion compound. Representative examples of the filamentous material are a plastic, a protein, a fiber, or a combination thereof. The filamentous material may comprise a plurality of ridges.

In a related aspect the attaching means comprises at least one spine. The spine may be one or more microneedles. Examples of the surgical fastener are a staple, a clip or a pin.

In another embodiment of this invention there is provided a surgical device comprising the activated surgical fastener described supra; an applicator to position at least one of the fastener in relation to at least two substrates; a means of delivering energy to the fastening means to effect a weld between the substrates; and a means to control the welding process. In all aspects of this embodiment the activated surgical fastener and the substrates are as described supra. Also in all embodiments the applicator may be positioned topically or endoscopically.

In one aspect of this embodiment the activated surgical fastener is a surgical suture. The applicator comprises a means of holding a first end and a second end of the suture in juxtaposition to one another to form a closed loop to effect the weld. The applicator further may comprise a means to apply tension to the ends of the suture.

In a related aspect the activated surgical fastener is a surgical fastener. The applicator may comprise means of applying pressure to position the surgical fastener. Pressure may be created with a spring mechanism or with a gas.

In this embodiment the means of delivering energy may be conductive or inductive. Examples of energy are radiofrequency energy, radiant energy or microwave energy. A preferable example is radiofrequency energy. Further in this embodiment the means to control the welding process may be electronic. A means to monitor changes in the activated surgical fastener, the substrates or both as a ferromagnetic material in the surgical fastening means reaches its Curie temperature is an example a control means. Alternatively, a feedback control circuit may be used to monitor voltage or current or the thermal history of the activated surgical fastener may be monitored.

In yet another embodiment of the present invention there is provided a method of surgically fastening at least two substrates comprising the steps of positioning the activated surgical fastener in relation to the substrates with the surgical device to attach the substrates; and forming a weld between the surgical fastener and said substrates with the surgical device thereby surgically fastening said substrates. In all aspects of this embodiment the activated surgical fastener, the substrates, the surgical device, including the positioning of the applicator of the surgical device, are as described supra.

As used herein, the term “weld” may be used interchangeably to represent bonding or attachment of one or more substrates including sections of tissue to another section of tissue or to attaching parts of a fastener such as a clip, pin or staple, to itself.

Provided herein are activated surgical fasteners, devices to position such fastener and methods to join biological tissues. The activated fastener are composed of materials or fusion composition that, upon activation, e.g., heating, will fuse with substrates such as tissues in order to produce a strong, uniform attachment or bond or weld. In the case of a surgical incision, the fastener may take the form of sutures, staples, pins, or clips. The fastener optionally may comprise one or more spines or ridges whereby such fasteners may be inserted into the tissues surrounding the wound.

A surgical suture may comprise a filamentous material and a fusion composition. The surgical fastener can be a staple, pin or clip and comprises a fusion composition. The fastener may be “activated” by electromagnetic energy, preferably in the radiofrequency range, but optionally in the optical or microwave range, to fuse with the tissues thus resulting in a weld. Upon activation the fusion compound will form a connection between two substrates, such as a folded tissue, a plurality of tissues, a graft or non-biological element with tissue.

The surgical sutures optionally may have substantially pointed ends and may comprise ridges. The surgical fasteners optionally may have spines or projections, such as serrations, ridges or raised edges. Such ridges or spines serve to increase friction between the fastener and the substrate thus temporarily holding the fastener in place while the welding process is taking place. The spines may be placed at angles relative to the substrate in order to achieve greater friction or locking of materials due to forces in opposing directions.

The fastener may comprise at least partially one or more of a protein, such as elastin or collagen, of an ionic species such as saline in a hydrogel, or of a ferromagnetic material. These are activated in the presence of electromagnetic energy such that the materials will bond or fuse to tissues thereby effecting a connection between the tissues. The fastener may also be or comprise a conductive element. The conductive element may be a fusion composition material, or integrated within a fusion composition, whereby the conductive element is inductively or conductively heated in order to activate it, i.e. generate heat in the system used for welding substrates together. Preferentially, the fastener is essentially composed of a biocompatible material.

The fastener may fasten two or more substrates such as a tissue, dressing, or graft, to a tissue whereby a conductive element is integrated within the fastener or in an applied fusion composition material. The conductive element is capable of transmitting energy for the purpose of connecting the substrates together. The element may optionally be removed after the tissue fixation treatment through physically withdrawing the element or through dissolving and absorption as a result of physiological processes.

The fastener may contain a mechanism for fastening tissues together that compresses the tissues upon activation. Such a mechanism of compression may include, for example, a hinge-like mechanism for clamping, an elastic material that shrinks upon activation and a shape memory alloy. At least one component of the mechanism has a different elasticity, changes configuration upon activation, or reacts to the applied energy with different characteristics than a second component in the device so that the tissues are compressed to each other.

The activated fastener may take advantage of the “skin effect” of induction heating and comprise different materials. The materials in the core may be somewhat shielded from heating occurring on the surface of the device. For example, it may be desirous to shield the interior of a fastener or suture that contains a pharmaceutical compound. By choosing the appropriate materials, frequency and power, the surface of these fastener may be preferentially heated, with little or no heating occurring in the core of the device, thereby providing some protection to the inner core which contains the pharmaceutical.

A device may be used to position at least one of the fasteners via an applicator to subsequently fasten two or more substrates, such as a tissue, dressing, or graft, to a tissue. The device may activate the fasteners by inductively or conductively delivering energy to the fasteners or sutures to weld these fasteners to the substrates. The device may further comprise a means to monitor the welding process. Optionally, the applicator may be made of such dimension as to fit inside a surgical trocar for endoscopic application.

The device may fix the fastener to tissue whereby a type of fusion composition containing a material composed at least in part of a ferromagnetic material is placed between the tissues or dressing to be connected. The fusion composition is heated by an external magnetic field until it reaches the Curie temperature of the ferromagnetic material. The heating ceases until the material cools below its Curie temperature whereupon the heating cycle can be repeated.

The applicator may be used to hold the suture ends in proximity to one another and in proximity to a conductive element and may incorporate a coil for inductive heating. Such an applicator may also be used endoscopically. The applicator has a means of holding two ends of a suture in place while fastening the substrates. The suture also may have ridges and a tubular component for locking of the suture in place. An applicator may comprise a ridged structure to complement a ridged suture to more firmly position and attach the suture to the substrates.

The ends of the sutures are juxtaposed to one another in opposite directions so that the welded area has a low and compact profile with respect to a surface to which they are attached. A tensioning and activation device may be applied to the suture ends in such manner as to secure the welds while positioned against the surface of the tissue to be secured. This aspect provides for a low-profile, high tension weld whereby it is not necessary to lift the suture above the tissue while applying tension and activation energy.

An applicator may comprise a forceps-like instrument to position and hold a suture to subsequently deliver energy to the suture to effect the weld. Furthermore, an applicator may hold the suture ends in place such that they are in proximity to an induction coil or conductive element. An energy generating mechanism present in the applicator, for example, an induction coil and an energy source, may be employed to weld or “activate” the suture once positioned by the applicator.

Furthermore, an applicator may load one or more fasteners such that pressure may be exerted to the fastener, either manually or by a pressure generating mechanism in the applicator, such that the fastener is made to attach to one or more substrates. An energy generating mechanism present in the applicator, for example, an induction coil and energy source, may be employed to activate the fasteners once in place. The applicator may preferably contain a mechanism to “load” additional fasteners automatically, allowing fasteners to be applied in succession.

The tissue welding process is monitored by changes in the electrical properties of the electromagnetic circuit that comprises the power supply, an induction coil, the material to be heated by the coil and the body. These changes may include, but not be limited to, changes in voltage or conductance or changes in the magnetic properties of a ferromagnetic material in a fusion composition as it reaches its Curie temperature. Alternatively, a feedback control circuit may be used to monitor voltage or current or the thermal history of the suture may be monitored with, for example, a thermocouple.

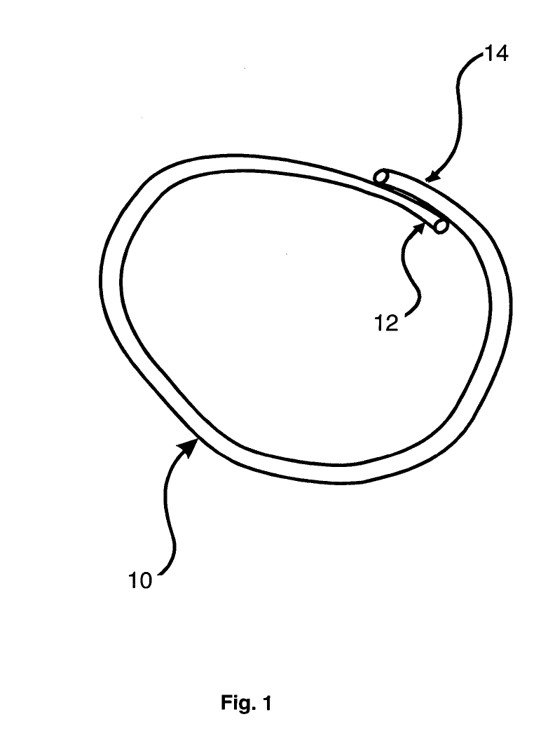

As described below, the invention provides a number of therapeutic advantages and uses, however such advantages and uses are not limited by such description. Embodiments of the present invention are better illustrated with reference to the FIGS. 1-7, however, such reference is not meant to limit the present invention in any fashion. The embodiments and variations described in detail herein are to be interpreted by the appended claims and equivalents thereof.

FIG. 1 depicts a length of surgical suture 10 having a cylindrical shape with a first end 12 and a second end 14. The surgical suture 10 is composed of a filamentous material and a metal (not shown) either distributed through the suture material or minimally present at the site of fixation. The first and second ends 12,14 are juxtaposed to one another in opposite directions such that the ends 12,14 form a weld upon activation.

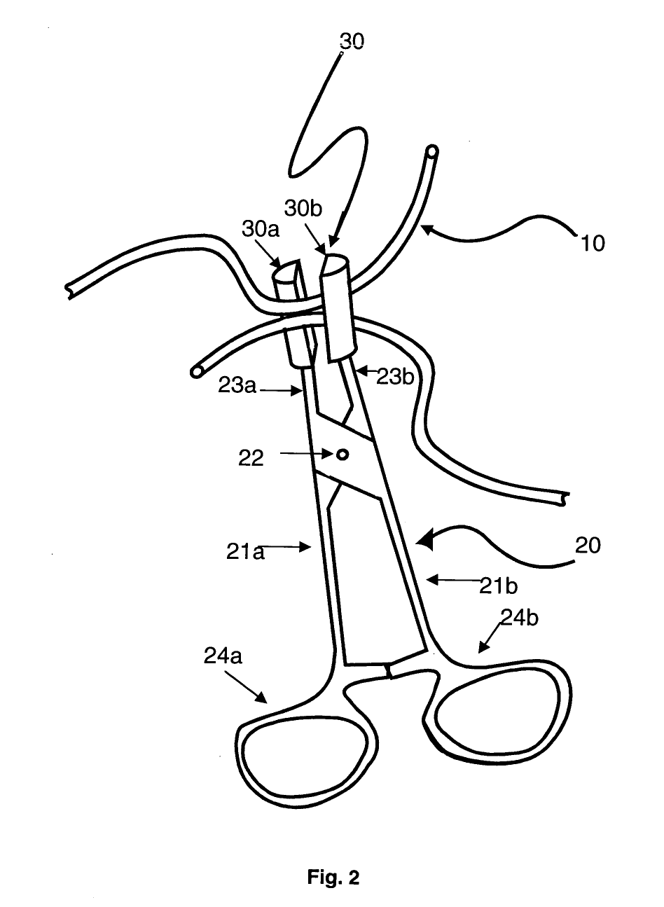

Continuing to refer to FIG. 1, FIG. 2 depicts a forceps-like surgical suture applicator 20. The suture applicator 20 comprises a scissors-like extension having two arms 21a,b pivotally connected at the center 22. The arms 21a,b have a first end 23a,b with elements 30a,b that transfers energy to two lengths of suture to be fixed 10 clamped therebetween and have a second end 24a,b comprising a gripping means. The elements 30a,b have an essentially planar inner surface and linearly extend from the first ends 23a,b such that the planar inner surfaces are juxtaposed in parallel relation when the applicator 20 is clamped. The pivotal action of the arms 21a,b increases or decreases the distance between the inner surfaces of the elements 30a,b such that the suture 10 may be positioned at a surgical site. The elements 30a,b are connected to an energy source (not shown).

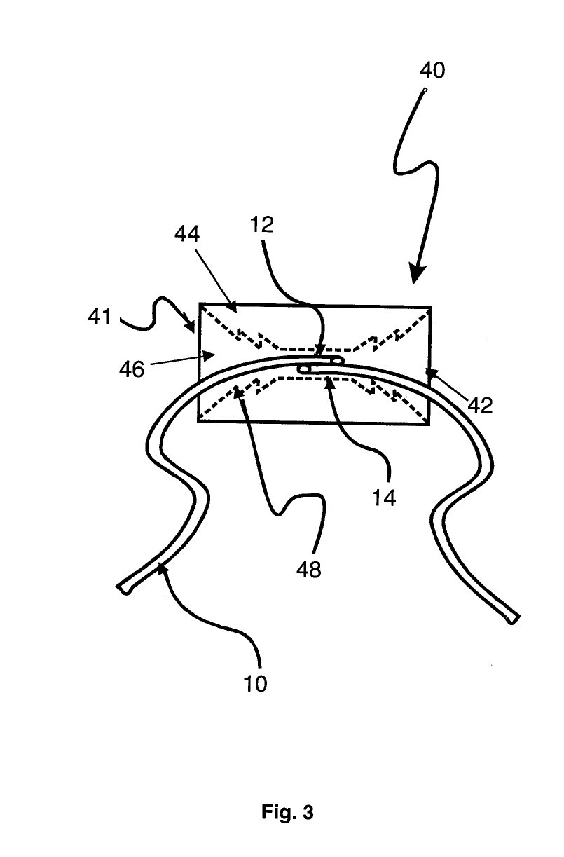

Continuing to refer to FIG. 1, FIG. 3 depicts a suture 10 having ends 12,14 that can be positioned within an applicator 40. The applicator 40 has a first end 41 and a second end 42 parallel to the first end 41 and a channel 46 on a surface 44 of the applicator 40 connecting the ends 41,42. A series of ridges 48 are disposed along the interior of the channel 46 such that the width of the channel 46 at the ends 12,14 is greater than the width of the channel 46 in the center of the applicator 40. The ends 12,14 of the suture 10 are inserted into the ends 41,42 of the applicator 40 until the ends 12,14 of the suture 10 overlap in the center of the channel 46. The ridges 48 hold the suture 10 taut while exposing the ends 12, 14 to applied energy. The applicator may be composed of two parts, separated by the channel 46, such that the applicator may be removed following fixation.

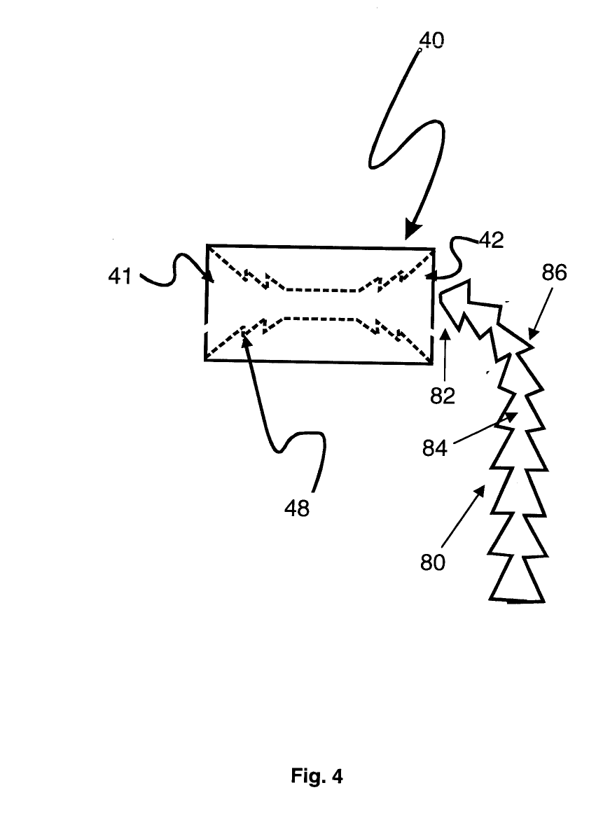

Continuing to refer to FIG. 3, FIG. 4 depicts a suture 80 inserted into the applicator 40. The suture 80 has a substantially pointed first end 82 and an outer surface 84 with a plurality of ridges 86 evenly distributed down the length of the suture 80. The first end 82 is inserted into one of the ends 41,42 of the applicator 40. The combination of the ridges 86 on the suture 80 and the ridges 48 on the applicator 40 holds the suture 80 in place. The applicator may be composed of two parts, separated by the channel, such that the applicator may be removed following fixation.

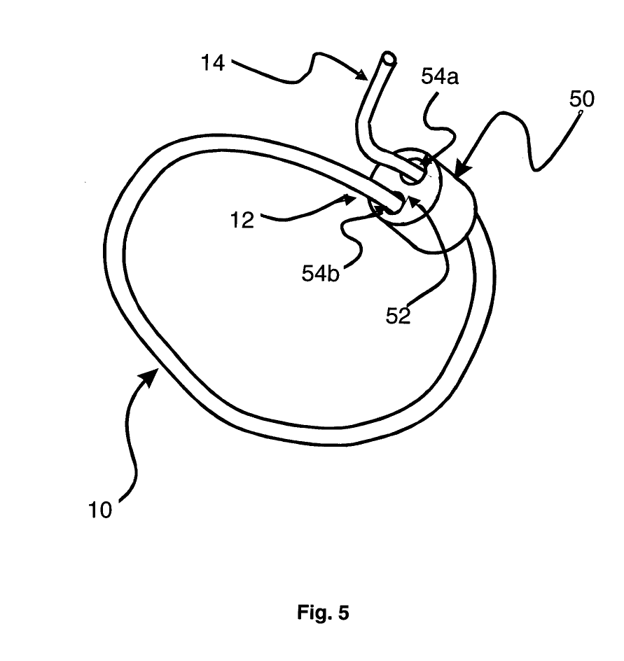

Continuing to refer to FIG. 1, FIG. 5 depicts an applicator 50 that holds the two ends 12,14 of a suture 10 in place while the applicator 50 is exposed to a magnetic field generated by an induction coil (not shown). The applicator 50 is cylindrical in shape with a first face 52 and a second face (not shown) parallel thereto. The first face 52 comprises two circular openings 54a,b positioned equidistant along a diameter thereof and the second face comprises a circular opening positioned as is opening 54a. An end 12 of a suture 10 is inserted into the applicator 50 via the opening 54a, exits the applicator 50 via the opening on the second face and is looped around to be inserted into opening 54b. The end 14 of the suture 10 is exterior to the applicator 50. This juxtaposes a selected two segments of the suture within the applicator. Application of a magnetic field to the applicator 50 effects a weld. The applicator may be composed of two parts, separated across the two circular openings 54a,b, such that the applicator may be removed following fixation.

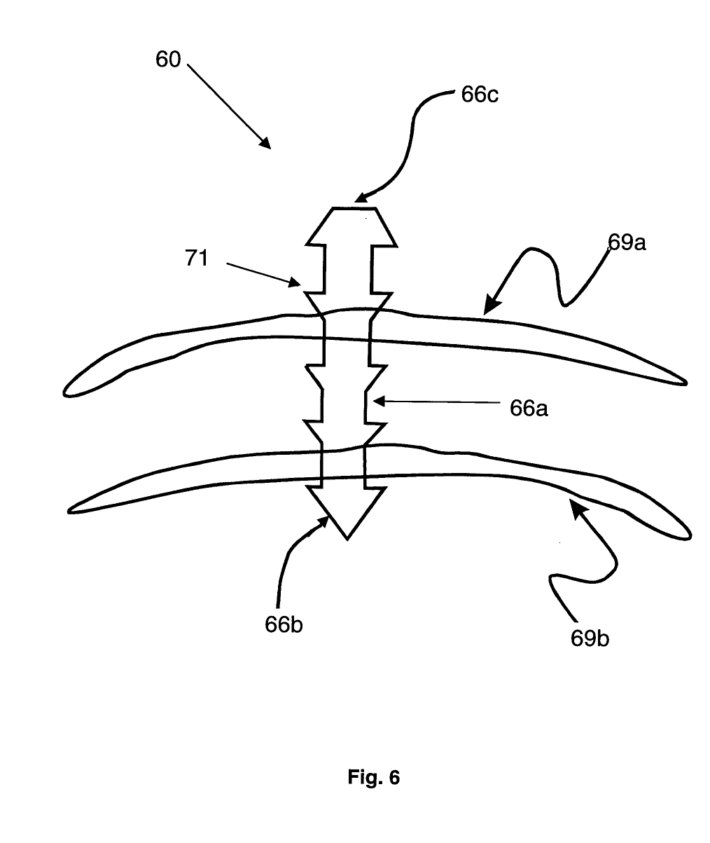

FIG. 6 depicts a surgical pin 60, composed at least in part of a fusion composition material, having a straight pin body 66a with a first pointed end 66b and a second truncated end 66c opposite the first end 66a. The first pointed end 66a on the surgical pin 60 provides ease of insertion into two overlapping sections of tissue 69a,b and anchors the surgical pin 60 at the outer surface of tissue segment 69b. The second truncated end 66c prevents the surgical pin 60 from completely piercing through the outer surface of tissue segment 69a upon pinning the overlapping tissue segments 69a,b together and anchors the surgical pin 60 to the outer surface of the tissue segment 69a. The pin body 66a has a plurality of spines 71 along the outer surface of the pin body 66a that provide friction or a temporary anchoring mechanism for placement between the two sections of overlapping tissue 66a,b.

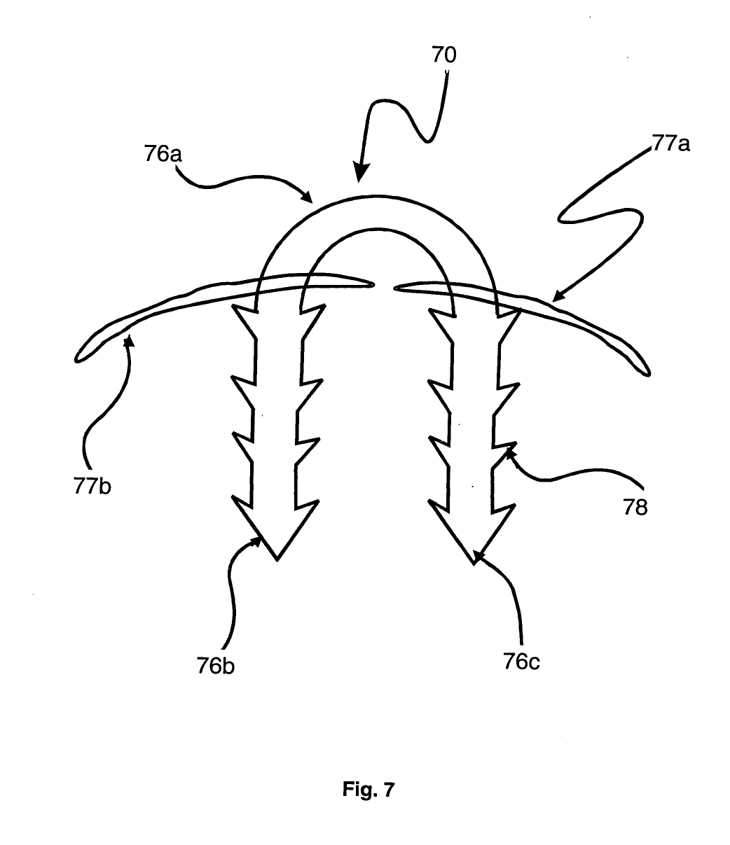

With reference to FIG. 6, FIG. 7 depicts a surgical staple 70, composed at least in part of a fusion composition material, having a symmetrically curved body 76a with pointed first and second ends 76b,c which is used to fasten tissues 77a,b which have been separated surgically or as the result of a wound. The surgical staple 70 has a plurality of spines 78 along the outer surface of the first and second ends 76b,c of the staple 70 that provide an anchoring mechanism for placement across the wound in the tissue 77a,b.

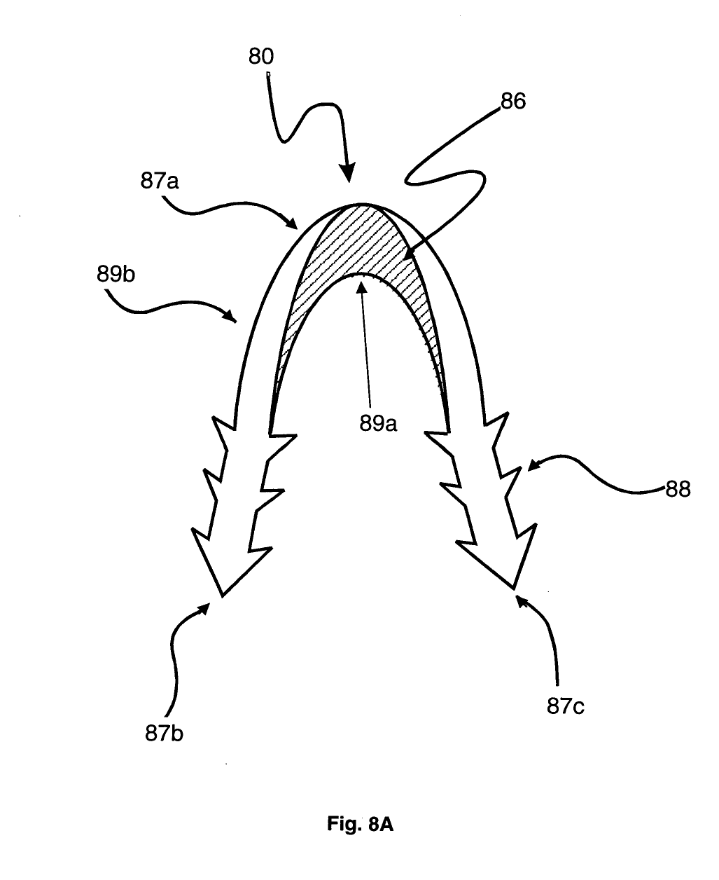

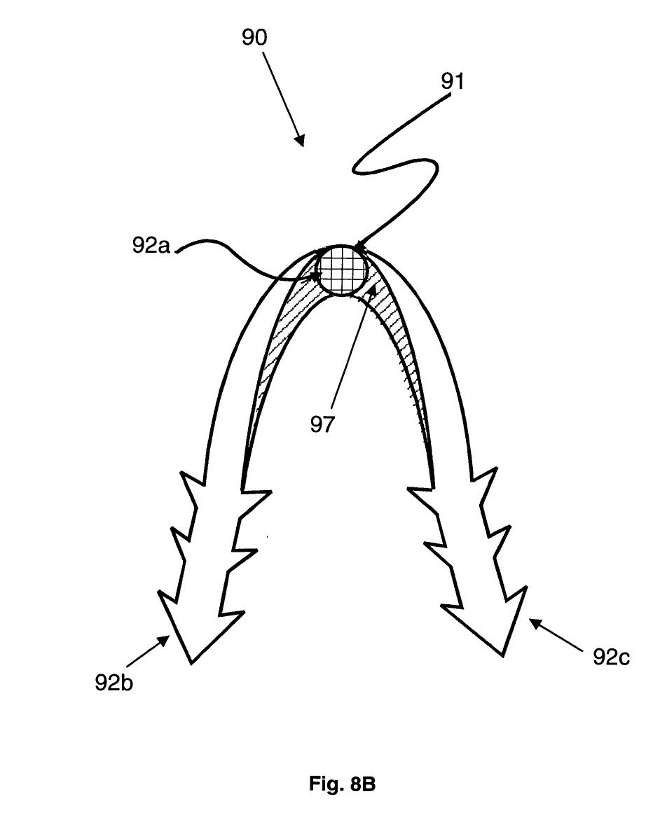

With reference to FIG. 7, FIGS. 8A and 8B depict embodiments of a surgical compression staple 80. In FIG. 8A the compression staple is a single piece composed at least in part of a fusion composition material, having a symmetrically curved body 87a with pointed first and second ends 87b,c and having a plurality of spines 88 along the outer surface of the first and second ends 87b,c of the staple 80 as in FIG. 7. The middle curved segment 87a of the compression staple 80 is comprised of an inner sleeve 86 of a flexible elastic polymer whereby the inner surface 89a of compression staple 80 is capable of greater shrinkage than the external surface 89b.

With reference to FIG. 8A, FIG. 8B depicts a variation thereof. FIG. 8A shows a multi-piece surgical compression staple further having a hinge 91 at the middle section 92a of the compression staple 90. Compression is effected by the hinging action on the two segments 92b,c of the compression staple 90 and by the inner sleeve of flexible elastic polymer 97 as in FIG. 8A.

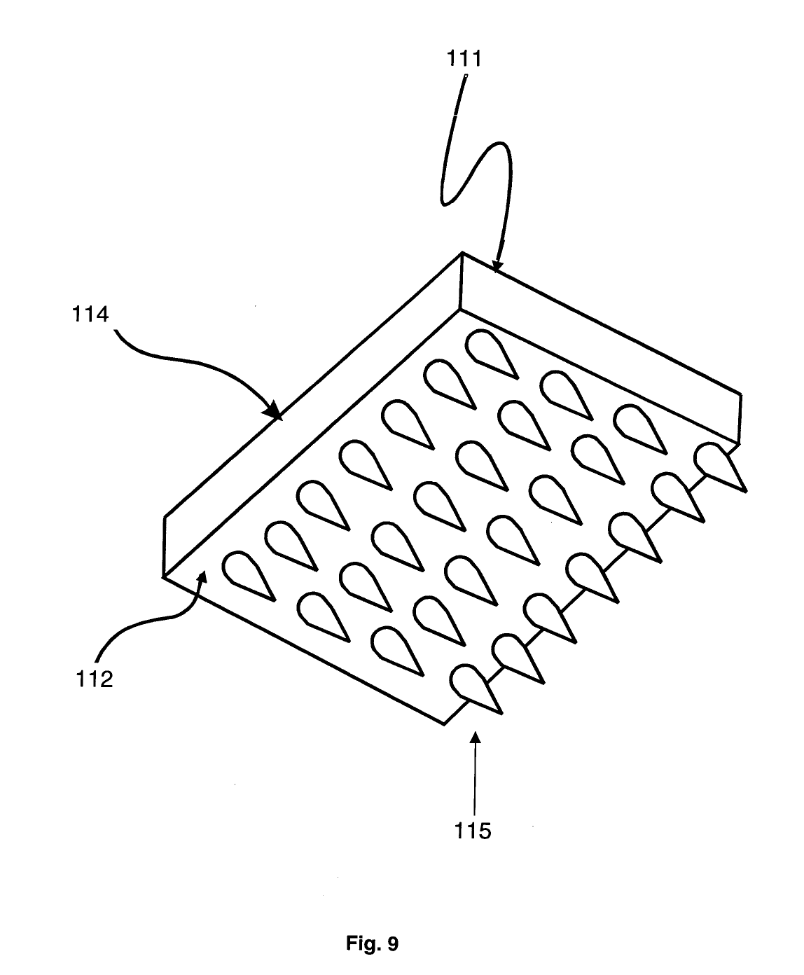

FIG. 9 depicts a tissue-fastening device 114, composed at least in part of a fusion composition material, having an outer surface 111 and inner surface 112. The inner surface 112 has a plurality of spines 115 disposed thereon and protruding from the inner surface 112 of the device 114 in a substantially perpendicular direction.

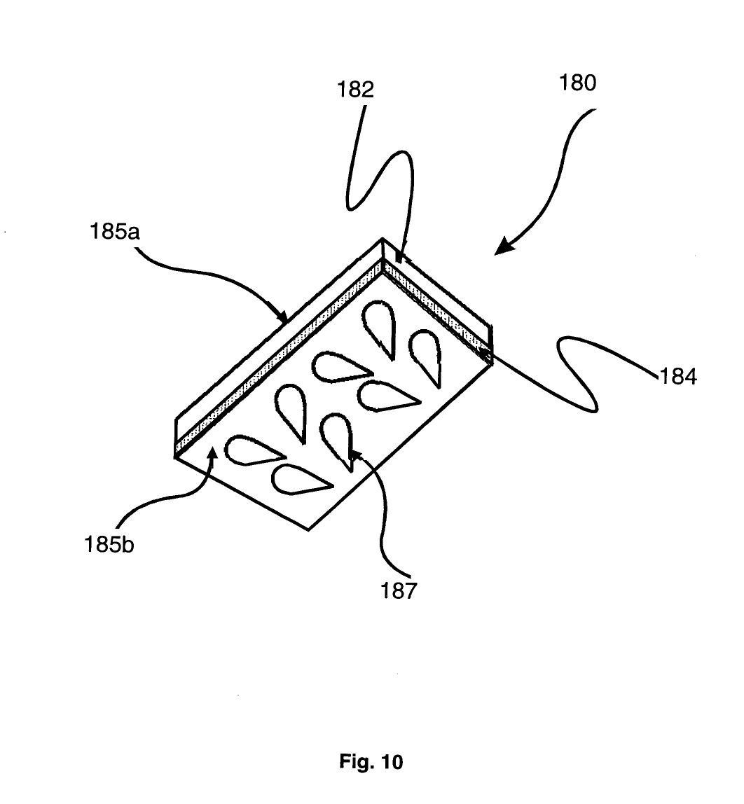

FIG. 10 depicts a compression style tissue-fastening device 180, composed at least in part of a fusion composition material, with layers 182,184 disposed one on the other having an outer surface 185a on outer layer 182 and inner surface 185b on inner layer 184. Layers 182, 184 may have different shape conforming qualities. The inner surface 185b has a plurality of angular spines 187 disposed thereon and protruding from inner surface 185b of inner layer 184 at varied angles.

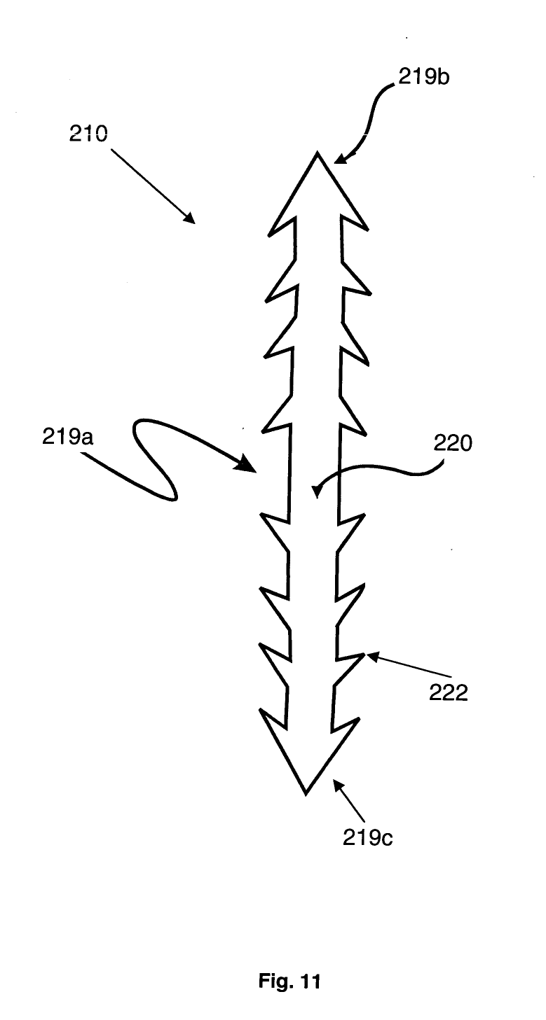

With reference to FIG. 6, FIG. 11 is a two-sided compression style tissue-fastening device 210, composed of a conductive, shrinkable material 220 and a fusion composition material, having a straight body 219a with a first pointed end 219b and a second pointed end 219c opposite the first end 219b. The device 210 has a plurality of angular spines 222 along the outer surface of the first and second ends 219b,c. One of each of the first or second ends 219b,c is placed and anchored in and between one of two sections of overlapping tissue (not shown). Shrinking the material 220 in combination with the spines 222 fastens and anchors the tissue sections together.

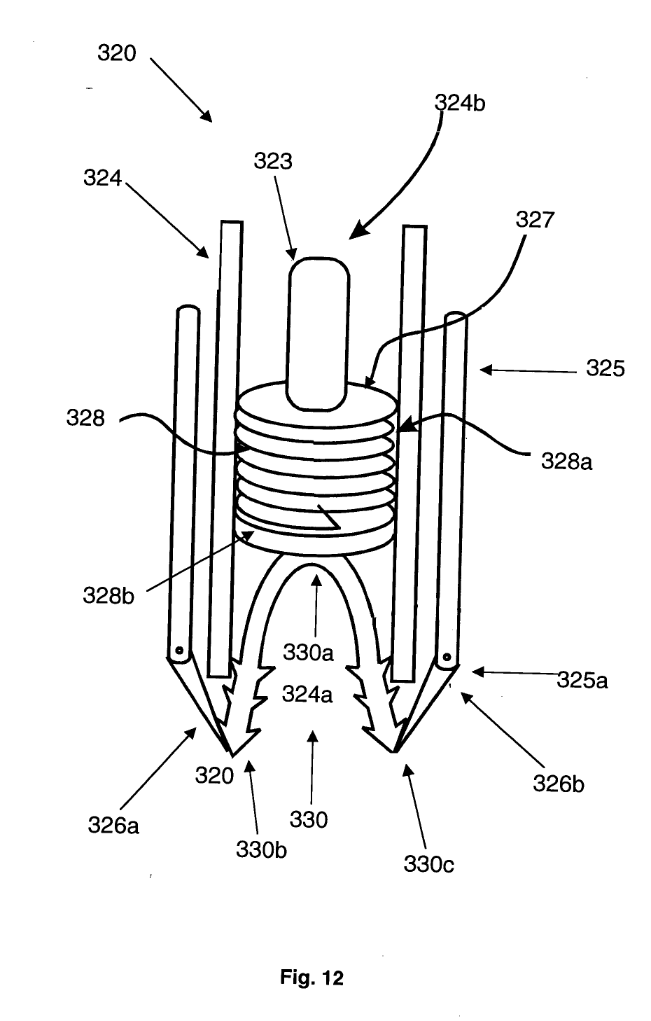

FIG. 12 depicts an applicator for fasteners 320. The applicator has an inner sleeve 324 with a first 324a open end and a second open end 324b and a retractable outer sleeve 325 with hinged restrainers 326 at a first open end 325a. The fastener 320 is positioned within the first open end 324a of the inner sleeve 324 such that the first end 330b and the second end 330c of the fastener 330 rest against the hinged restrainers 326a,b and the curved middle section 330a of the fastener 330 is positioned against the lower end 328b of a spring mechanism 328 disposed within the inner sleeve 324. A plunger 323 applies pressure to the upper end 328a of the spring mechanism 328 which positions the fastener 330 through force applied to a disk 327 at the upper end 328a of the spring mechanism 328. The action of the force on the disk 327 compresses the spring mechanism 328 and transfers the downwardly applied force to the fastener 330. The first and second ends 330b,c of the fastener 330 simultaneously are forced past the hinged restrainers 326a,b and can thus be positioned within at least one tissue (not shown).

One skilled in the art will appreciate readily that the present invention is well adapted to carry out the objects and obtain the ends and advantages mentioned, as well as those objects, ends and advantages inherent herein. The present examples, along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. An activated surgical fastener to effect a weld between at least two substrates in an individual, comprising:
a means for attaching said substrates; and
a fusion composition to weld said attached substrates upon the application of energy thereto.

2. The activated surgical fastener of claim 1, wherein said energy is applied conductively or inductively.

3. The activated surgical fastener of claim 1, wherein energy applied to said fusion composition is radiofrequency energy, radiant energy or microwave energy.

4. The activated surgical fastener of claim 3, wherein energy applied to said fusion composition is radiofrequency energy.

5. The activated surgical fastener of claim 1, further comprising at least one conductive element.

6. The activated surgical fastener of claim 5, wherein said conductive element comprises at least one electrode.

7. The activated surgical fastener of claim 6, wherein said conductive element is an electrode array, said array comprising a plurality of isolated electrode terminals.

8. The activated surgical fastener of claim 6, wherein said conductive element is a material capable of being heated inductively.

9. The activated surgical fastener of claim 1, further comprising at least one material wherein said material changes configuration upon the application of said energy.

10. The activated surgical fastener of claim 9, wherein said material is a shape-memory polymer or a shape-memory metal.

11. The activated surgical fastener of claim 9, wherein said material(s) shrink upon heating.

12. The activated surgical fastener of claim 1, wherein said attaching means is a surgical suture comprising:
a filamentous material having a first end and a second end such that said first and second ends are juxtaposed to form a closed loop for attaching said substrates, said ends welded upon application of energy to said fusion compound.

13. The activated surgical fastener of claim 12, wherein said filamentous material comprises a plastic, a protein, a fiber, or a combination thereof.

14. The activated surgical fastener of claim 12, wherein said filamentous material comprises a plurality of ridges.

15. The activated surgical fastener of claim 1, wherein said attaching means comprises at least one spine.

16. The activated surgical fastener of claim 15, wherein said spine is one or more microneedles.

17. The activated surgical fastener of claim 15, wherein the attaching means is a staple, a clip or a pin.

18. The activated surgical staple, a clip or a pin of claim 1, wherein said fusion composition is biocompatible.

19. The activated surgical fastener of claim 1, wherein said fusion composition comprises a protein, a metal, a ferromagnetic material, a conducting polymer, a pharmaceutical, an ionic mixture or a combination thereof.

20. The activated surgical fastener of claim 1, wherein the substrate is a biological tissue.

21. The activated surgical fastener of claim 20, wherein said tissue is surgically separated or is surgically grafted.

22. The activated surgical fastener of claim 1, wherein said fastener is positioned in relation to said substrates by an applicator.

23. The activated surgical fastener of claim 22, wherein said applicator utilizes pressure to position said fastener.

24. The activated surgical fastener of claim 23, wherein said pressure is created with a spring mechanism or with a gas.

25. The activated surgical fastener of claim 22, wherein said applicator is used endoscopically.

26. A surgical device, comprising:
the activated surgical fastener means of claim 1;
an applicator to position at least one of said fasteners in relation to at least two substrates;
a means of delivering energy to said fastener to effect a weld between said substrates; and,
a means to control the welding process

27. The surgical device of claim 26, wherein said activated surgical fastener is a surgical suture and said applicator comprises a means of holding a first end and a second end of said suture in juxtaposition to one another to form a closed loop to effect said weld.

28. The surgical device of claim 27, said applicator further comprising a means to apply tension to the ends of said suture.

29. The surgical device of claim 26, wherein said applicator comprises a means of applying pressure to position said surgical fastener to effect said weld.

30. The surgical device of claim 29, wherein said pressure is created with a spring mechanism or with a gas.

31. The surgical device of claim 26, wherein said means of delivering energy is conductive or inductive.

32. The surgical device of claim 31, wherein said energy is radiofrequency energy, radiant energy or microwave energy.

33. The surgical device of claim 32, wherein said energy is radiofrequency energy.

34. The surgical device of claim 26, wherein said means to control the welding process is electronic.

35. The surgical device of claim 26, wherein said means to control the welding process comprises:
a means to detect changes in said surgical fastening means, in said substrates or both as a ferromagnetic material comprising said surgical fastening means reaches a Curie temperature.

36. The surgical device of claim 26, wherein said means to control the welding process comprises:
a feedback control circuit to monitor voltage or current.

37. The surgical device of claim 26, wherein said means to control the welding process comprises a means to monitor the thermal history of the surgical fastener.

38. The surgical device of claim 26, wherein said applicator is positioned topically or endoscopically.

39. The device of claim 26, wherein at least one said substrates is a biological tissue.

40. The device of claim 39, wherein said tissue is surgically separated or surgically grafted.

41. A method of surgically fastening at least two substrates comprising the steps of:
positioning the activated surgical fastener of claim 26 in relation to said substrates with the surgical device of claim 26 to attach said substrates; and
forming a weld between said surgical fastener and said substrates with the surgical device of claim 26 thereby surgically fastening said substrates.

42. The method of claim 41, wherein said activated surgical fastener is positioned topically or via an endoscope.

43. The method of claim 41, wherein at least one of the substrates is a biological tissue.

44. The method of claim 43, wherein said tissue is surgically separated or is surgically grafted.
